(19) United States
(12) Patent Application Publication
(10) Pub. No.: US 2011/0259348 A1
(43) Pub. Date: Oct. 27, 2011

(54) EAR PADDING FOR OXYGEN TUBING

(76) Inventor: DARLA SCHMIDT, Deerfield, MI (US)

(21) Appl. No.: 12/765,102

(22) Filed: Apr. 22, 2010

(51) Int. Cl. A61F 11/14

(52) U.S. Cl. 128/866

(57) ABSTRACT

The present invention relates to padding for placement over a section of oxygen tubing comprising: a section of hypo-allergenic foam; and a slit along the length of the foam, where the slit provides a means to insert a section of oxygen tubing and the foam conforms to the outer surface of the tubing. The padding provides protection for the user of the oxygen tubing.

### BACKGROUND OF THE INVENTION

1. Field of Invention

The present invention relates to flexible ear pads that are inserted over oxygen tubing.

2. Description of Related Art

Many individuals require oxygen as part of their healthcare maintenance. Oxygen is usually delivered through tubing where the tube loops around the patient's ears and supplies an outlet of oxygen near the patient's breathing nostrils. The oxygen tubing is referred to as nasal cannula or oral nasal cannula where a flexible tube with short, multiple end branches comfortably insert into the nostrils of the patient. The tubing may be placed over the patient's ears therefore over a period of time, where the tubing may cause lesions, sores or swelling to a patient's ears. Consequently it would be advantageous to have a means or a device that could alleviate the swelling or soreness that may occur through the prolonged use of oxygen tubing over a patient's ears.

### SUMMARY OF THE INVENTION

The present invention relates to padding for placement over a section of oxygen tubing comprising: a section of hypo-allergenic foam; and a slit along the length of the foam, where the slit provides a means to insert a section of oxygen tubing and the foam conforms to the outer surface of the tubing. The padding provides protection for the user of the oxygen tubing.

### BRIEF DESCRIPTION OF DRAWINGS

FIG. 1 depicts a section of oxygen tubing that includes ear pads according to the present invention.

FIG. 2A shows a detailed view of the ear padding inserted over a section of oxygen tubing according to the present invention.

FIG. 2B depicts a reverse view of the ear padding according to the present invention.

### DETAILED DESCRIPTION

The present invention relates to ear padding that is conveniently inserted over the oxygen tubing to protect and pad the patient's ear from abrasion due to continued use of the oxygen tubing. The oxygen tubing is looped over a patient's ear in order to place the oxygen outlets in close proximity to the patient's breathing path. The present invention includes a hypo-allergenic foam that is used to pad the ear when using the oxygen tubing. The hypo-allergenic foaming reduces the rubbing and abrasion that occurs through prolonged use of the oxygen tubes over a patient's ears.

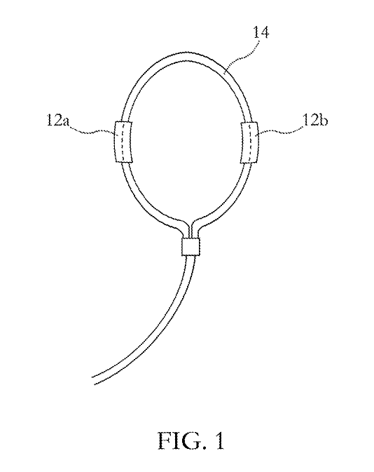

FIG. 1 depicts the insertion of foam pads 12a, 12b at opposite sides of an oxygen tube 14. As depicted, a foam pad 12a, 12b is placed on each side of the oxygen tubing to protect each ear of the patient. The oxygen tubing 14 is of a conventional size. The foam pads 12a, 12b each include a slit that allows for the insertion and removal of the foam padding onto and off of the oxygen tubing 14. The foam pads 12a, 12b are cylindrical in shape to conform to the tubing.

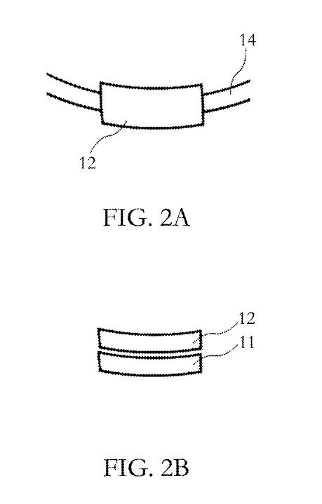

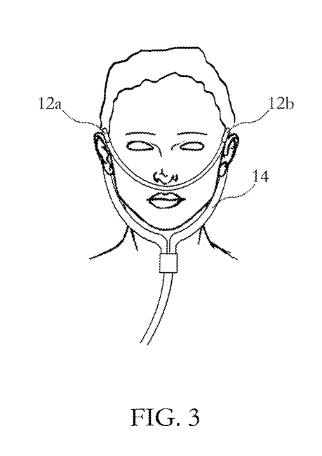

FIG. 2A depicts an exemplary foam ear pad 12 according to the present invention. The ear pad 12 inserts over the oxygen tube 14. The reverse view of the foam pad is depicted in FIG. 2B. A slit 11 is provided in the foam pad 12 to allow for the insertion of the foam pad 12 over the oxygen tubing. The foam 12 is soft, flexible and does not interfere with the flow of oxygen through the tubing but however provides sufficient padding to alleviate discomfort by the patient. In one particular embodiment, the foam pad 12 is about three inches in length and is inserted over the tubing and placed near the rear ear region to protect the ear from discomfort.

The ear protection foam attachment according to the present invention is capable of being inserted onto various sizes of oxygen tubes and is suitably adjusted to the patient's ears. The foam ear pads may be removed and washed as needed to prevent the accumulation of dust, grease and dirt. Also the ear pads may be discarded and replaced with a new pair due to the ease in which the padding may be removed and placed upon the oxygen tubing. The instant invention has been shown and described in what it considers to be the most practical and preferred embodiments. It is recognized, however, that departures may be made there from within the scope of the invention and that obvious modifications will occur to a person skilled in the art.

What is claimed is:

1. Padding for placement over an oxygen tube comprising:
a. a section of hypo-allergenic foam; and
b. a slit along the length of the foam, where the slit provides a means to insert a section of oxygen tubing and the foam conforms to the outer surface of the tubing.

2. The padding for placement according to claim 1, where the section of hypo-allergenic foam is cylindrical in shape.
